(19) United States
(12) Patent Application Publication
(10) Pub. No.: US 2011/0231945 A1
(43) Pub. Date: Sep. 22, 2011

(54) GOLD EVENING PRIMROSE AND METHOD OF BREEDING THE SAME

(76) Inventor: Bong Ho Kim, Seoul (KR)

(21) Appl. No.: 12/671,692

(22) PCT Filed: Jul. 31, 2008
(86) PCT No.: PCT/KR2008/004459

§ 371 Date: Feb. 1, 2010

(30) Foreign Application Priority Data
Feb. 5, 2009 (KR) 1020070076818

(51) Int. Cl. A01H 5/00; A01H 1/02

(52) U.S. Cl. 800/260; 800/323

(57) ABSTRACT

Disclosed is a gold evening primrose, a novel variety of evening primrose. The gold evening primrose has a height of 20 to 40 cm, panicle-type uniflorous large yellow flowers blooming during the day, and leaves tinged with reddish purple in winter enduring a low temperature of −15° C. The gold evening primrose adapts well to a variety of environments, develops large flowers, blossoms during the day, has high ornamental value due to long blooming period ranging from early June to late July, exhibits superior fertility, is resistant to harmful insects, can be semi-permanent Iy gardened through a single planting, thereby reducing costs, and has evergreen leaves that are tinged with red in winter, thus being suitable for use in landscaping and gardening throughout all four seasons.

### TECHNICAL FIELD

The present invention relates to a gold evening primrose, a new variety of evening primrose and a method for breeding the same.

### BACKGROUND ART

Evening primroses are a species of the genus Oenothera, native to South America and Chile and perennial plants naturalized and grown throughout South Korea. The plants have a height of about 150 cm, comparable to the human waist, the stems are erect and the leaves alternate. The flowers are yellow, develop singly from the axils of the leaves, and open in the evening and close in the morning while turning slightly red. The flower has four sepals wherein two respective sepals are joined, and curl downward upon blooming. The flower has four petals. The plants blossom in midsummer and grow well even in sterile environments.

However, conventional Evening primroses are tall, have small flowers, bloom in the evening, and are wholly invisible on the ground in winter, thus disadvantageously being unsuitable for use in floriculture and gardening.

### DISCLOSURE

### Technical Problem

Therefore, the present invention has been made in view of the above problems, and it is one object of the present invention to provide a Gold evening primrose, as an improved new variety of conventional Evening primroses unsuitable for use in floriculture and gardening.

It is another object of the present invention to provide a method for breeding the Gold evening primrose.

### Technical Solution

In accordance with an exemplary embodiment of the present invention, the above and other objects can be accomplished by the provision of a gold evening primrose, a novel variety of evening primrose, having a height of 20 to 40 cm, panicle-type uniflorous large yellow flowers blooming during the day, and leaves tinged with reddish purple in winter while withstanding temperatures as low as −15° C.

In accordance with another exemplary embodiment of the present invention, provided is a method for breeding a gold evening primrose, comprising: hybridization-breeding the pistil of large-flower evening primrose (Oenothera lamarckiana) and the stamen of island evening primrose (Oenothera sp.) to produce seeds F1, disseminating the seeds F1 and then selecting a first superior individual from the seeds F1; hybridization-breeding the pistil of the first superior individual and the stamen of island evening primrose to produce seeds F2, disseminating the seeds F2 and then selecting a second superior individual from the seeds F2; and subjecting varieties originated from the selection of the second superior individual to division/propagation.

### Advantageous Effects

The present invention provides a uniform new variety of gold evening primrose different from conventional evening primroses. The gold evening primrose adapts well to a variety of environments, develops large flowers, blossoms during the day, has high ornamental value due to long blooming period ranging from early June to late July, exhibits superior fertility, is resistant to harmful insects, can be semi-permanently gardened through a single planting, thereby reducing costs, and has perennial leaves being tinged with red in winter, thus being suitable for use in landscaping and gardening for all four seasons.

### DESCRIPTION OF DRAWINGS

The above and other objects, features and other advantages of the present invention will be more clearly understood from the following detailed description taken in conjunction with the accompanying drawings, in which:

FIG. 1 is a flow chart illustrating a process for breeding the gold evening primrose;

FIG. 2 is an image illustrating the gold evening primrose according to one embodiment of the present invention;

FIG. 3 is an image illustrating the gold evening primrose having reddish purple leaves in winter according to one embodiment of the present invention; and

FIG. 4 is a notice of registration verifying the fact that the gold evening primrose according to one embodiment of the present invention is registered as a new variety on the KOREA SEED & VARIETY SERVICE.

### BEST MODE

The gold evening primrose according to one embodiment of the present invention can be bred through hybridization and varietal selection.

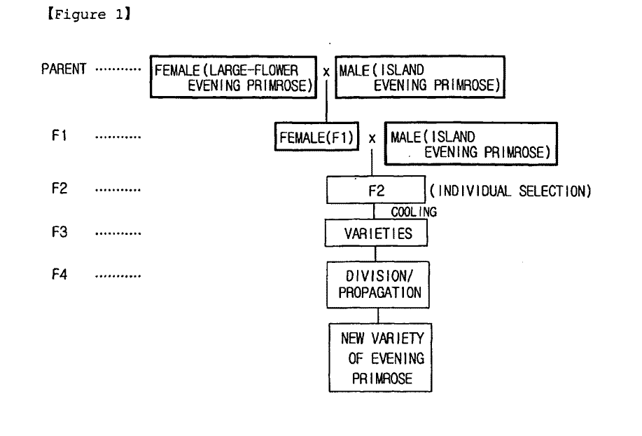

A detailed description of a process for breeding gold evening primrose is given in FIG. 1.

Referring to FIG. 1, the pistil of large-flower evening primrose (Oenothera lamarckiana) and the stamen of island evening primrose are subjected to hybridization-breeding to produce seeds F1. After the seeds F1 are disseminated, a first superior individual is selected therefrom. The pistil of the first superior individual and the stamen of island evening primrose are subjected to hybridization-breeding to produce seeds F2. After the seeds F2 are disseminated, a second (short) superior individual is selected therefrom and then planted. Among the resulting plants, varieties which have wide petals and are prolific (underground procumbent-type) are subjected to division and propagation.

At this time, the seeds F2 may be treated at a low temperature of −5 to −12° C. for about one week to produce more uniform seeds.

The scientific/specific name of Gold evening primrose is Oenothera odorata and cultivated variety name thereof is “Hwang geum”.

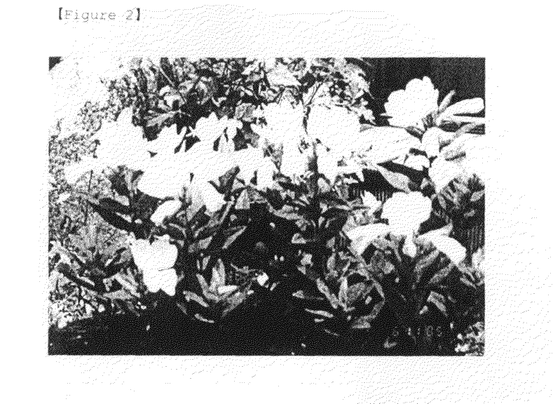

FIG. 2 is an image illustrating the gold evening primrose according to one embodiment of the present invention.

Referring to FIG. 2, the gold evening primrose is short and has anthocyanin-free stems. The color of the stems is green.

In addition, the gold evening primrose has the following leaf characteristics: arrangement type: decussate; leaf color: single; leaf shape: arrow; leaf apex: cleft; leaf margin: palmatifld; leaf length: 3-4 cm; leaf width: medium; venation: palmate venation; and spots: absent.

The gold evening primrose has the following flower characteristics: inflorescence type: panicle; flower type: uniflorous; color: yellow (single), inflorescence direction: upward; flower width: medium; number of petals: 4; and spots: absent.

The blooming period of the gold evening primrose ranges from June to July.

Conventional evening primroses are tall, about 150 cm in height, but the gold evening primrose is short, 20 to 40 cm in height and thus exhibits superior effects upon use in floriculture and gardening. In addition, the gold evening primrose develops large beautiful flowers and thus has high ornamental value, as compared to conventional evening primroses. The gold evening primrose blooms during the day, whereas conventional evening primroses bloom in the evening and are thus unsuitable for landscaping and gardening. In addition, the gold evening primrose blooms over a long blooming period of up to 90 days, depending on soil moisture level, endures against humid soil and has superior soil coating effects.

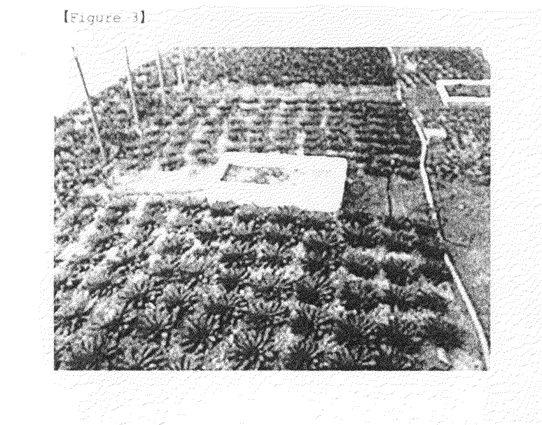

FIG. 3 is an image illustrating the gold evening primrose having reddish purple leaves in winter according to one embodiment of the present invention. Referring to FIG. 3, the leaves of the gold evening primrose are evergreen, turn reddish purple in winter, and can withstands temperatures of −15° C. or below. On the other hand, conventional evening primroses are rhizocarp, wherein the leaves above the ground die in winter, thus being wholly invisible on the ground.

Conventional evening primroses can be grown wild together with weeds, but the gold evening primrose grows smaller than weeds, thus requiring weeding. In addition, seed-bearing of the gold evening primrose is difficult to realize in the wild and requires specific treatment. The gold evening primrose has rhizome thus hardly causes damage to other plants.

The gold evening primrose requires sufficient watering to prevent soil and its roots from being dried upon division and cultivation of the species. The gold evening primrose is free from damage by blight and harmful insects so long as it has good permeability, and may be involved in a problem wherein ladybugs nibble away the leaves in September and October, but can avoid the problem by preventing breeding of the ladybirds using an insecticide. When the gold evening primrose is bred along with regular continuous watering, since it is grown well in moisturized soil, high-quality seedlings can be mass-produced.

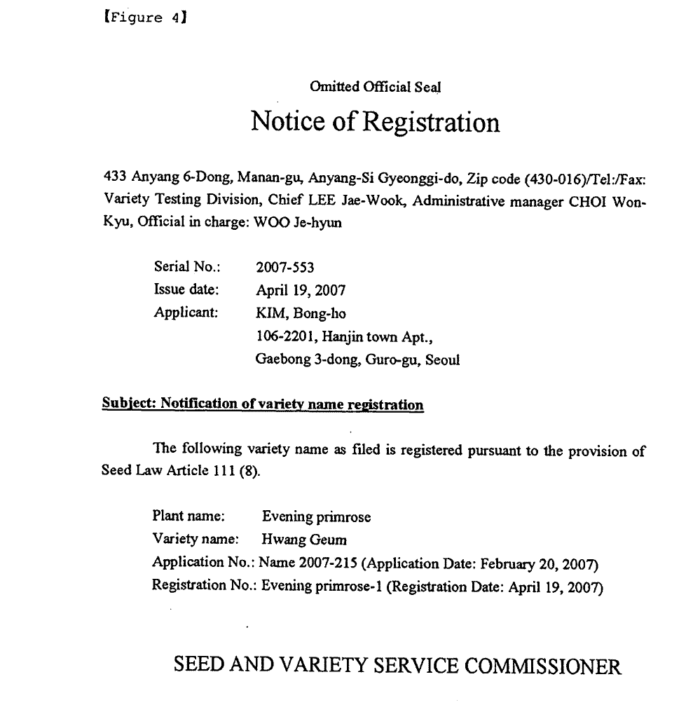

FIG. 4 shows a notice of registration verifying the fact that the gold evening primrose according to one embodiment of the present invention is registered as a new variety on the KOREA SEED & VARIETY SERVICE. As can be confirmed from the certificate, the gold evening primrose is registered as a new variety on the KOREA SEED & VARIETY SERVICE.

### MODE FOR INVENTION

A process for breeding the gold evening primrose according to one embodiment of the present invention will be described below.

In 1999, large-flower evening primrose (Oenothera lamarckiana) (♀) and island evening primrose (Oenothera sp.) (♂), which grew naturally in Wando of South Korea, were subjected to hybridization-breeding to produce seeds F1. In 2000, after the seeds F1 were disseminated, a first superior individual was selected therefrom. The first superior individual (♀) and island evening primrose (♂) were subjected to hybridization-breeding to produce seeds F2. After the seeds F2 were disseminated, in the process of selecting a second superior individual, varieties were found. In 2003 and 2004, the varieties were vegetatively reproduced to obtain a novel evening primrose plant, referred to as a “gold evening primrose”.

### INDUSTRIAL APPLICABILITY

The gold evening primrose according to one embodiment adapts itself well to environments, develops large flowers, blossoms during the day and has a long blooming period ranging from early June to late July, thus having high ornamental values.

In addition, the gold evening primrose has leaves which do not fall even in winter and are tinged with reddish purple, thus being suitable for use in landscaping and gardening throughout four seasons.

1. A gold evening primrose, a novel variety of evening primrose, having:
a height of 20 to 40 cm,
panicle-type uniflorous large yellow flowers blooming during the day, and
perennial leaves tinged reddish purple in winter and enduring a low temperature of −15° C.

2. A method for breeding a gold evening primrose, comprising:
hybridization-breeding the pistil of large-flower evening primrose (Oenothera lamarckiana) and the stamen of island evening primrose (Oenothera sp.) to produce seeds F1, disseminating the seeds F1 and then selecting a first superior individual from the seeds F1;
hybridization-breeding the pistil of the first superior individual and the stamen of island evening primrose to produce seeds F2, disseminating the seeds F2 and then selecting a second superior individual from the seeds F2; and
subjecting varieties originated from the selection of the second superior individual to division/propagation.
